(19) United States
(12) Patent Application Publication
(10) Pub. No.: US 2023/0102735 A1
(43) Pub. Date: Mar. 30, 2023

(54) SURGICAL AUGMENTED REALITY WORKSTATION

(71) Applicant: Medivis, Inc., New York, NY (US)

(72) Inventors: Long Qian, Brooklyn, NY (US); Christopher Morley, New York, NY (US); Osamah Choudhry, New York, NY (US)

(21) Appl. No.: 17/489,679

(22) Filed: Sep. 29, 2021

(51) Int. Cl. A61B 34/10; A61B 34/20; A61B 34/00; G06T 19/00; A61B 50/13; G02B 27/01

(52) U.S. Cl. CPC A61B 34/10; A61B 34/20; A61B 34/25; G06T 19/006; A61B 50/13; G02B 27/017

(57) ABSTRACT

Various embodiments of an apparatus, methods, systems and computer program products described herein are directed to a workstation cart. One or more embodiments of the workstation cart (“workstation”) include a monitor, a first module, a second module and a third module connected to main support beam. An Augmented Reality (AR) headset device holder is disposed upon the first module. One or more AR headset device charging cables are connected to the second module. At least one computer system and a router are located inside the first module. A power unit and/or a battery(s) are located inside the third module.

### BACKGROUND

Conventional systems provide for the rendering of virtual reality (VR) and Augmented Reality (AR) environments. Such environments provide a visualization of various portions of the physical world as well as simulations of certain events that will or may occur in the physical world. These conventional systems include communication with input devices controlled by one or more users. The input devices allow the users to select certain types of actions and activities within the rendered environments. In many cases, these environments rendered by conventional systems may be specific to certain types of industries.

### SUMMARY

Various embodiments of an apparatus, methods, systems and computer program products described herein are directed to a workstation cart. The workstation cart described herein provides advantages over convention systems by providing mobility to a user accessing virtual reality (VR) and Augmented Reality (AR) environments. For example, the workstation cart may provide a surgeon accessing an AR surgical environment in the operating room.

One or more embodiments of the workstation cart (“workstation”) include a monitor, a first module, a second module and a third module connected to main support beam. An Augmented Reality (AR) headset device holder is disposed upon the first module. At least one computer system and a router are located inside the first module. One or more AR headset device charging cables are connected to the second module. A power unit and/or a battery(s) are located inside the third module.

A method of configuring a workstation comprises attaching a monitor, a first module, a second module and a third module to a main support beam. Attaching an Augmented Reality (AR) headset device holder upon the first module and one or more AR headset device charging cables to the second module. Placing at least one computer system and a router inside the first module and placing at least one of: a power unit and one or more batteries inside the third module.

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1 is a diagram illustrating an exemplary environment in which some embodiments may operate.

FIG. 2 is a diagram illustrating an exemplary method that may be performed in some embodiments.

FIG. 3 is a diagram illustrating an exemplary environment in which some embodiments may operate.

FIG. 4 is a diagram illustrating an exemplary environment in which some embodiments may operate.

FIG. 5 is a diagram illustrating an exemplary environment in which some embodiments may operate.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments include a computer system(s). A computer system may include a processor, a memory, and a non-transitory computer-readable medium. The memory and non-transitory medium may store instructions for performing methods and steps described herein.

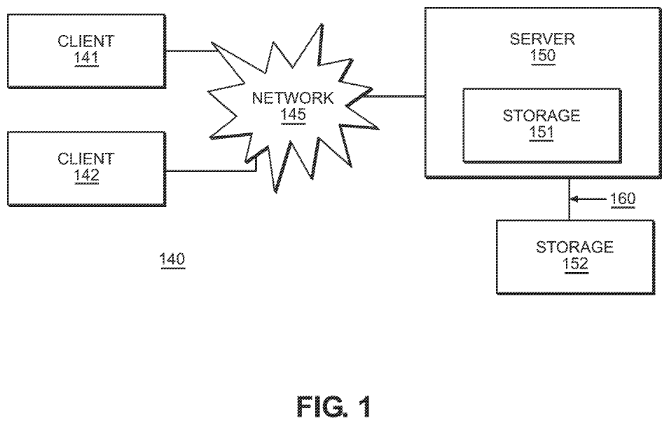

A diagram of exemplary network environment with which embodiments may operate (or communicate) is shown in FIG. 1. In the exemplary environment 140, two clients 141, 142 are connected over a network 145 to a server 150 having local storage 151. Clients and servers in this environment may be computers. Server 150 may be configured to handle requests from clients.

The exemplary environment 140 is illustrated with only two clients and one server for simplicity, though in practice there may be more or fewer clients and servers. The computers have been termed clients and servers, though clients can also play the role of servers and servers can also play the role of clients. In some embodiments, the clients 141, 142 may communicate with each other as well as the servers. Also, the server 150 may communicate with other servers.

The network 145 may be, for example, local area network (LAN), wide area network (WAN), telephone networks, wireless networks, intranets, the Internet, or combinations of networks. The server 150 may be connected to storage 152 over a connection medium 160, which may be a bus, crossbar, network, or other interconnect. Storage 152 may be implemented as a network of multiple storage devices, though it is illustrated as a single entity. Storage 152 may be a file system, disk, database, or other storage.

In accordance with the above discussion, embodiments can be used to store a file on local storage such as a disk or on a removable medium like a flash drive, CD-R, or DVD-R. Furthermore, embodiments may be used to store a file on an external storage device connected to a computer over a connection medium such as a bus, crossbar, network, or other interconnect. In addition, embodiments can be used to store a file on a remote server or on a storage device accessible to the remote server.

Furthermore, cloud computing is another example where files are often stored on remote servers or remote storage systems. Cloud computing refers to pooled network resources that can be quickly provisioned so as to allow for easy scalability. Cloud computing can be used to provide software-as-a-service, platform-as-a-service, infrastructure-as-a-service, and similar features. In a cloud computing environment, a user may store a file in the “cloud,” which means that the file is stored on a remote network resource though the actual hardware storing the file may be opaque to the user.

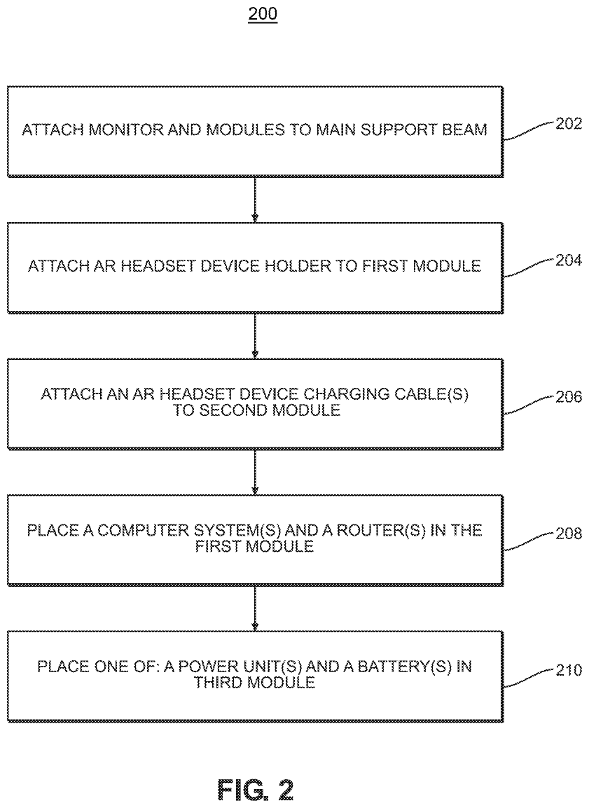

As shown in the flowchart 200 of FIG. 2, various embodiments of the workstation may be configured by attaching a monitor, a first module, a second module and a third module to a main support beam. (Act 202). Attaching an Augmented Reality (AR) headset device holder upon the first module. (Act 204) Attaching one or more AR headset device charging cables to the second module. (Act 206) Placing at least one computer system and a router inside the first module (Act 208) and placing at least one of: a power unit and one or more batteries inside the third module. (Act 210) It is understood that various embodiments of the workstation may be configured according to Acts 202, 204, 206, 208, 210 performed in any ordered sequence.

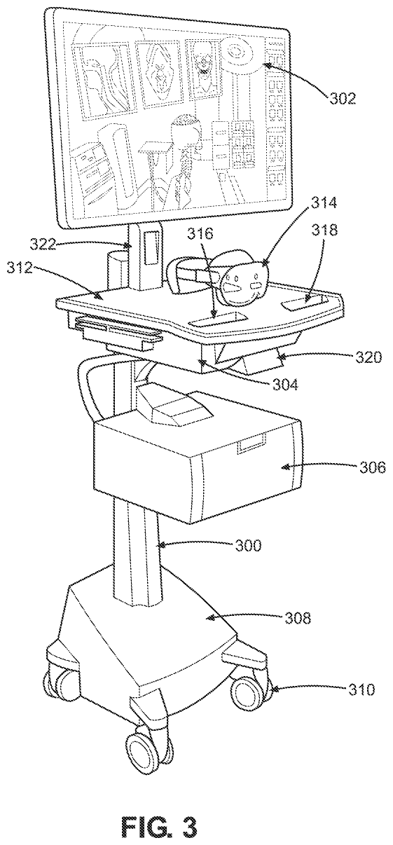

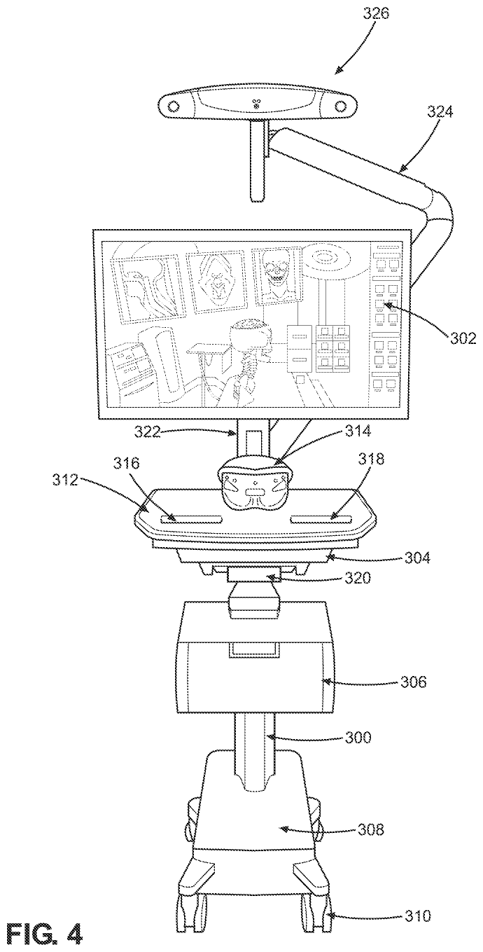

As shown in FIGS. 3 and 4, the various embodiments of the workstation include a main support beam 300. For example, the main support beam may be vertically oriented and may further include an internal hollow portion suitable for the storage of one or more electrical cables. A touchscreen monitor(s) 302, a first module 304 and a second module 306 may be attached to the main support beam 300. For example, the first module 304 and the second module 306 may both be situated as being perpendicular to the main support beam 300 such that each model 304, 306 extends away from the main support beam 300. A terminal portion of the main support beam 304 connects to a third module 308. One or more wheels 310 may further be connected to a portion(s) of the third module 308. A power unit and/or one or more batteries may be stored inside the third module 308. A charging cable may be connected to the power unit inside the third module 308.

In some embodiments, the one or more spatial audio speakers may be attached to the touchscreen monitor 302. In some embodiments, an extension arm 324 may be connected to the main support beam 300. An external tracking device 326 may further be disposed on the extension arm. For example, the external tracking device 326 may be a single or dual, RBG or RGBD or infrared sensor. The external tracking device 326 may also be an electromagnetic tracking device.

The touchscreen monitor 302 may be connected to the main support beam 300 via a height adjustable structure 322. The height adjustable structure 322 may be programmable such that the height adjustable structure 322 may be programed to alternate between various configurations associated with one or more predefined touchscreen monitor heights.

In various embodiments, the first module 304 contains one or more computer systems and one or more routers. In various embodiments, a 5G modem may be stored inside the first module 304. A flat surface 312 may be horizontally disposed upon the first module 304. A device holder 314 may further be disposed on the surface 312. In various embodiment s, the device holder 314 may be shaped to be compatible with a shape of an AR headset device. For example, a user wearing an AR headset device may remove the AR headset device from the user's head and place the AR headset device on the device holder 314. The device holder 314 holds the AR headset device in place while the workstation is stationary or being moved. For example, the device holder 314 may be molded according to a shape that is compatible with a shape of at least a portion of a VR headset device as well. The flat surface 312 includes one or more handle cut-outs 316, 318 which can be grabbed by a user in order to move the workstation in various directions. An external CD-ROM may be accessible via the first surface 312.

In some embodiments, one or more USB ports for external data reading may further be accessible via the first surface 312. In addition, the computer system(s) stored inside the first module 304 may include a USB-C port for tethering with one or more AR headset devices. The USB-C port may also be accessible via the first surface 312 as well.

The first module 304 includes a button panel 320. The button panel 320 may include a power control button that can turn on and turn off all electrically powered portions, modules, units and components of the workstation. The power control button may further turn on and turn off, individually, the touchscreen monitor 302, the computer system(s) housed inside the first module 304 as well as the 5G modem further disposed on the workstation.

Other types of buttons included on the button panel 320 include one or more of: a button for restarting the computer system(s) housed inside the first module 304, a button to adjust a height of the touchscreen monitor(s) 302 between various preconfigured heights and a button to turn on and turn off a video input and a button to turn on and off an outport included in the workstation. The button panel 320 further includes one or more indicators to represent a respective power state of the entire workstation, the computer system(s) in the first module 304 and touchscreen monitor(s) 302.

The second module 306 may be a storage module. The second module 306 may be defined according to internal dimensions suitable for the storage of one or more inactive AR headset device s (and/or one or more inactive VR headset devices). The second module 306 may include one or more charging cables that can connect to the respective stored, inactive AR headset devices. The second module 306 may further be suitable for the storage of one or more wireless ultrasound probes and a medical instrument(s) that has one or more fiducial markers.

In some embodiments, various ports may also be disposed on the workstation. For example, an ethernet connection may be disposed on the workstation for connecting to a network, such as an internal hospital network. A frame grabber may be included on the workstation for grabbing video data from various types of available modalities. The workstation may further include one or more HDMI/DVI video input portsand one or more HDMI/DVI video output ports.

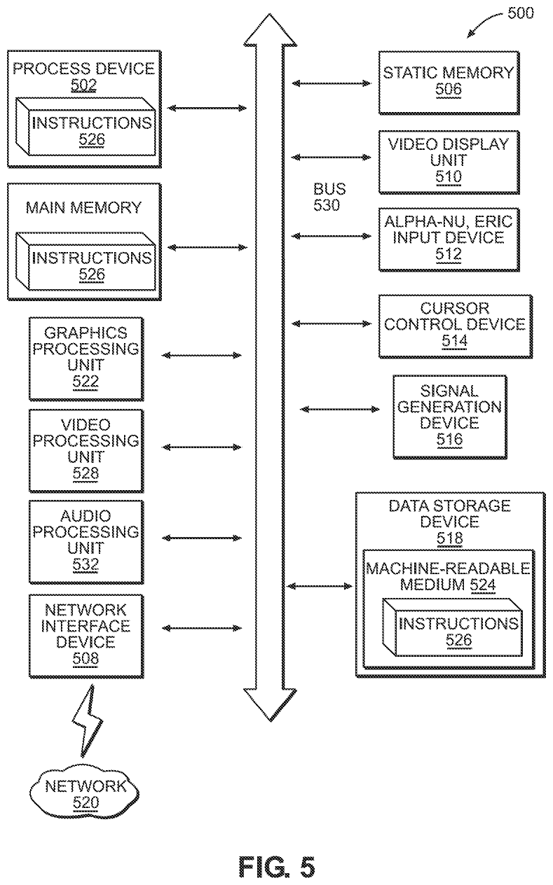

FIG. 5 illustrates an example of a computer system. In alternative implementations, the computer system may be connected (e.g., networked) to other machines in a LAN, an intranet, an extranet, and/or the Internet. The computer system may operate in the capacity of a server or a client computer system in client-server network environment, as a peer computer system in a peer-to-peer (or distributed) network environment, or as a server or a client computer system in a cloud computing infrastructure or environment.

The computer system may be a personal computer (PC), a tablet PC, a set-top box (STB), a Personal Digital Assistant (PDA), a cellular telephone, a web appliance, a server, a network router, a switch or bridge, or any computer system capable of executing a set of instructions (sequential or otherwise) that specify actions to be taken by that computer system. Further, while a single computer system is illustrated, the term “computer system” shall also be taken to include any collection of computer systems that individually or jointly execute a set (or multiple sets) of instructions to perform any one or more of the methodologies discussed herein.

The example computer system 500 includes a processing device 502, a main memory 504 (e.g., read-only memory (ROM), flash memory, dynamic random access memory (DRAM) such as synchronous DRAM (SDRAM) or Rambus DRAM (RDRAM), etc.), a static memory 506 (e.g., flash memory, static random access memory (SRAM), etc.), and a data storage device 518, which communicate with each other via a bus 530.

Processing device 502 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 502 may also be one or more special-purpose processing devices such as an application specific integrated circuit (ASIC), a field programmable gate array (FPGA), a digital signal processor (DSP), network processor, or the like. The processing device 502 is configured to execute instructions 526 for performing the operations and steps discussed herein.

The computer system 500 may further include a network interface device 508 to communicate over the network 520. The computer system 500 also may include a video display unit 510 (e.g., a liquid crystal display (LCD) or a cathode ray tube (CRT)), an alphanumeric input device 512 (e.g., a keyboard), a cursor control device 514 (e.g., a mouse), a graphics processing unit 522, a signal generation device 516 (e.g., a speaker), graphics processing unit 522, video processing unit 528, and audio processing unit 532.

The data storage device 518 may include a machine-readable storage medium 524 (also known as a computer-readable medium) on which is stored one or more sets of instructions or software 526 embodying any one or more of the methodologies or functions described herein. The instructions 526 may also reside, completely or at least partially, within the main memory 504 and/or within the processing device 502 during execution thereof by the computer system 500, the main memory 504 and the processing device 502 also constituting machine-readable storage media.

In one implementation, the instructions 526 include instructions to implement functionality corresponding to the components of a device to perform the disclosure herein. While the machine-readable storage medium 524 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the computer system and that cause the computer system to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a computer system (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A method of configuring a workstation cart, comprising:
attaching a monitor, a first module, a second module and a third module to a main support beam;
attaching an Augmented Reality (AR) headset device holder upon the first module;
attaching one or more AR headset device charging cables to the second module;
placing at least one computer system and a router inside the first module; and
placing at least one of: a power unit and one or more batteries inside the third module.

2. The method of claim 1, wherein the second module comprises internal dimensions compatible for storage of one or more AR headset devices.

3. The method of claim 1, further comprising: placing a button panel on the first module.

4. The method of claim 3, wherein the button panel includes one or more indicators for indicating a power status of one or more of: the workstation cart, the computer system in the first module and the monitor.

5. The method of claim 1, wherein the monitor comprises a touchscreen monitor.

6. The method of claim 5, further comprising: attaching one or more spatial audio speakers to the touchscreen monitor.

7. The method of claim 6, further comprising: connecting an external tracking device to the main support beam.

8. The method of claim 7, further comprising: connecting the touchscreen monitor to the main support beam via a programmable height adjustable structure.

9. The method of claim 1, further comprising: connecting a charging cable to the power unit inside the third module.

10. The method of claim 10, further comprising: attaching one or more wheels to the third module.

11. The method of claim, further comprising:
attaching the monitor above the first module;
attaching the first module above the second module; and
attaching the second module above the third module.

12. A workstation cart comprising:
a monitor, a first module, a second module and a third module connected to main support beam;
an Augmented Reality (AR) headset device holder disposed upon the first module;
one or more AR headset device charging cables connected to the second module;
at least one computer system and a router inside the first module; and
at least one of: a power unit and one or more batteries inside the third module.

13. The workstation cart of claim 12, wherein the second module comprises internal dimensions compatible for storage of one or more AR headset devices.

14. The workstation cart of claim 12, further comprising: a button panel on the first module.

15. The workstation cart of claim 14, wherein the button panel includes one or more indicators for indicating a power status of one or more of: the workstation cart, the computer system in the first module and the monitor.

16. The workstation cart of claim 12, wherein the monitor comprises a touchscreen monitor.

17. The workstation cart of claim 16, further comprising: one or more spatial audio speakers attached to the touchscreen monitor.

18. The workstation cart of claim 16, further comprising: an external tracking device connected to the main support beam.

19. The workstation cart of 16, further comprising: a programmable height adjustable structure connecting the touchscreen monitor to the main support beam.

20. The workstation cart of 12, further comprising:
a charging cable connected to the power unit inside the third module; and
one or more wheels attached to the third module.
